(19) United States
(12) Patent Application Publication
(10) Pub. No.: US 2006/0275336 A1
(43) Pub. Date: Dec. 7, 2006

(54) Preparation of an osteoinductive agent

(75) Inventor: Tjaart Andries Du Plessis, Pretoria (ZA)

Correspondence Address: NIXON & VANDERHYE, PC, 901 NORTH GLEBE ROAD, 11TH FLOOR, ARLINGTON, VA, 22203, US

(73) Assignees: DE VILLIERS, Malan, Pretoria (ZA); Tjaart Andries Du Plessis

(21) Appl. No.: 10/557,192

(22) PCT Filed: May 18, 2004
(86) PCT No.: PCT/IB04/50736

§ 371 Date: Jul. 31, 2006

(30) Foreign Application Priority Data
May 19, 2003 (ZA) 2003/1344

(51) Int. Cl. A61F 2/00

(52) U.S. Cl. 424/423; 422/22; 604/82

(57) ABSTRACT

This invention relates to a method for the preparation of an osteoinductive agent, the use of such an agent, and to a kit for preparing such an agent. This invention further relates to the use of the said kit in the preparation and dispensing of such an osteoinductive agent in a method of reconstructive bone surgery. The kit includes an osteoinductive agent comprising a modified naturally occurring biocompatible biopolymer which was subjected, in the solid, or dry state, to a source of ionising radiation in the presence of a mediating gas and annealed in the absence of oxygen at a temperature of from 40° C. to 120° C. to render the product in a dry particulate form, the product being disposed in a hermetically sealed container containing oxygen-free gas.

### INTRODUCTION AND BACKGROUND TO THE INVENTION

This invention relates to a method for the preparation of an osteoinductive agent, the use of such an agent, and to a kit for preparing such an agent. This invention further relates to the use of the said kit in the preparation and dispensing of such an osteoinductive agent in a method of reconstructive bone surgery.

It is known to use demineralised bone (DMB) in a biopolymer carrier such as hyaloronic acid (HA) or collagen, as an osteoinductive agent in reconstructive bone surgery. The biopolymer of some of these systems are chemically cross-linked and a first disadvantage of such osteoinductive systems is that they require both the DMB and the associated biopolymer carrier to be prepared under aseptic conditions and dispensed from a customised hypodermic syringe to ensure the sterile presentation of the osteoinductive agent during the surgical procedure.

Further disadvantages of the known osteoinductive systems are that they are prepared and stored in the form of a wet putty in modified hypodermic syringes and have to be radiation sterilised and kept at −40° C. to prevent any biological or radiation breakdown of the systems. It is difficult to store and handle osteoinductive systems at such low temperatures and the integrity of the systems could be jeopardised should the cold chain be broken.

In this specification, the term biopolymer includes within its scope a polymer derived from a biological source, whether plant, microorganism or animal.

### OBJECT OF THE INVENTION

It is therefore an object of the present invention to provide a method for the preparation of an osteoinductive agent, the use of such an agent, a kit for preparing such an agent, and the use of the said kit in the preparation and dispensing of such an osteoinductive agent, with which the aforesaid disadvantages can be overcome or at least minimised.

### SUMMARY OF THE INVENTION

According to a first aspect of the invention there is provided a method for the preparation of an osteoinductive agent including the steps of:
modifying a naturally occurring biocompatible biopolymer by subjecting the biopolymer in the solid, or dry state, to a source of ionising radiation in the presence of a mediating gas; and
annealing the resulting product in the absence of oxygen at a temperature of from 40° C. to 120° C. to render the product in a dry particulate form;
thereafter removing any residual mediating gas; and
disposing the product in a hermetically sealed container containing oxygen-free gas.

The naturally occurring biocompatible biopolymer may be selected from the group consisting of collagen; hyaloronic acid; demineralised bone (DMB); and mixtures thereof.

The method may, in the case of the said mixtures, include the further steps of first subjecting the biocompatible biopolymers separately from each other to the said source of ionising radiation in the presence of the said mediating gas; and thereafter mixing the irradiated biocompabble biopolymers.

Alternatively, the method may, in the case of the said mixtures, include the further steps of first mixing the biocompatible biopolymers; and thereafter subjecting the mixture to the said source of ionising radiation in the presence of the said mediating gas.

The biocompatible biopolymer may be subjected to a minimum absorbed irradiation dose of 16 kGy.

The hermetically sealed container may be a secondary container and the method may Include the further step of disposing the product inside a first primary container, which is disposed within the hermetically sealed secondary container.

The method may include the further step of providing the first primary container in the form of a syringe—type container, having a plunger for dispensing the contents thereof and an outlet opening having a diameter larger than 0.6 mm, to allow for the dispensing of the said product in a relatively viscous form.

The method may include the further step of filling the space in the first primary container not occupied by the product with the said oxygen-free gas.

The method may include the further steps of providing a second primary container also in the form of a syringe—type container; disposing liquid in the second primary container, and disposing the second primary container in the hermetically sealed secondary container.

The method may include the further step of providing the said liquid in the form of pyrogen-free water.

The method may include the further step of filling the secondary container with oxygen-free gas and capturing the oxygen-free gas inside the hermetically sealed secondary container.

The method may include the further step of disposing the hermetically sealed secondary container inside a hermetically sealed tertiary container.

The method may include the further step of filling the tertiary container with oxygen-free gas and capturing the oxygen-free gas inside the hermetically sealed tertiary container.

The method may include the further steps of subjecting the said containers and their contents, in kit form, to a terminal radiation sterilisation process.

Preferably, the sterilisation process includes the step of subjecting the containers and their contents to a minimum absorbed irradiation dose of 25 kGy.

The method may include the further step of opening the sealed containers and mixing the said liquid with the said product in a dry particulate form to hydrate the product to form an osteoinductive agent in the form of a pliable viscous putty.

The method may include the further step of dispensing the osteoinductive agent from the first primary container to a bone reconstruction site.

The method may include the further step of providing the oxygen-free gas in an inert form.

The method may include the further step of providing the said inert oxygen-free gas in the form of nitrogen.

According to a second aspect of the invention there is provided a kit for preparing and dispensing an osteoinductive agent including a modified naturally occurring biocompatible biopolymer which was subjected, in the solid, or dry state, to a source of ionising radiation in the presence of a mediating gas and annealed in the absence of oxygen at a temperature of from 40° C. to 120° C. to render the product in a dry particulate form, the product being disposed in a hermetically sealed container containing oxygen-free gas.

The naturally occurring biocompatible biopolymer may be selected from the group consisting of collagen; hyaloronic acid; demineralised bone (DMB); and mixtures thereof.

In the case of the said mixture, the biocompatible biopolymers may be subjected separately from each other in the presence of the said mediating gas to the said source of ionising radiation and thereafter be mixed.

Alternatively, in the case of the said mixture, the biocompatible biopolymers may first be mixed and thereafter be subjected to the said source of ionising radiation in the presence of the said mediating gas.

The biocompatible biopolymers may be subjected to a minimum absorbed irradiation dose of 16 kGy.

The sealed container may be a secondary container and the product may be disposed inside a first primary container, which is disposed within the hermetically sealed secondary container.

The first primary container may be in the form of a syringe—type container, having a plunger for dispensing the contents thereof and an outlet opening having a diameter larger than 0.6 mm, to allow for the dispensing of the product in a relatively viscous form.

The space in the primary container not occupied by the product may be filled with the said oxygen-free gas.

The kit may include a second primary container containing a liquid and being disposed in the hermetically sealed secondary container.

The liquid may be in the form of pyrogen-free water.

The hermetically sealed secondary container may be disposed inside a hermetically sealed tertiary container.

The tertiary container may be filled with oxygen-free gas.

The secondary and tertiary containers may each be vacuum formed from a radiation stable, gas—impermeable material.

The secondary and tertiary containers may be closed by a closure comprising at least one layer of a radiation stable, gas—impermeable material.

Preferably the closure comprises a tri-laminate of an aluminium layer sandwiched between an internal layer of polyethylene and an outer layer of polyester.

Further according to the invention, the said containers are subjected, in kit form, to a terminal radiation sterilisation process at a minimum absorbed radiation dose from 10 to 80 kGy, preferably 25 kGy.

According to a third aspect of the invention there is provided an osteoinductive agent prepared in accordance with the method of the first aspect of the invention.

According to a fourth aspect of the invention there is provided a method of reconstructive bone surgery in humans or animals including the steps of:
providing the kit in accordance with the second aspect of the invention;
opening the secondary and tertiary containers;
hydrating the said dry particulate product by injecting the sterile liquid into the first primary container and mixing the liquid and the product to form an osteoinductive putty;
dispensing the putty into a bone reconstruction site from the first primary container; and
closing the site to allow bone reconstruction to take place.

Further according to the invention the above steps take place under aseptical conditions.

### BRIEF DESCRIPTION OF THE DRAWINGS

The invention will now be described further by way of a non-limiting example 20 with reference to the accompanying drawings, wherein:

FIG. 1 is an exploded perspective view of a kit in accordance with a preferred embodiment of the invention for use in the preparation and dispensing of an osteoinductive agent;

FIG. 2 is an assembled perspective view of the kit of FIG. 2;

FIG. 3 is a plan view of a set of first primary containers, for use in kits similar to those of FIG. 1, containing a biocompatible biopolymer in dry or solid form and disposed in a hermetically sealed pouch containing a mediating gas, being subjected to a source of ionising radiation; and

FIG. 4 is a side view, in use, of a first and second primary containers illustrating a step in the preparation of the said osteoinductive agent.

### DESCRIPTION OF A PREFERRED EMBODIMENT OF THE INVENTION

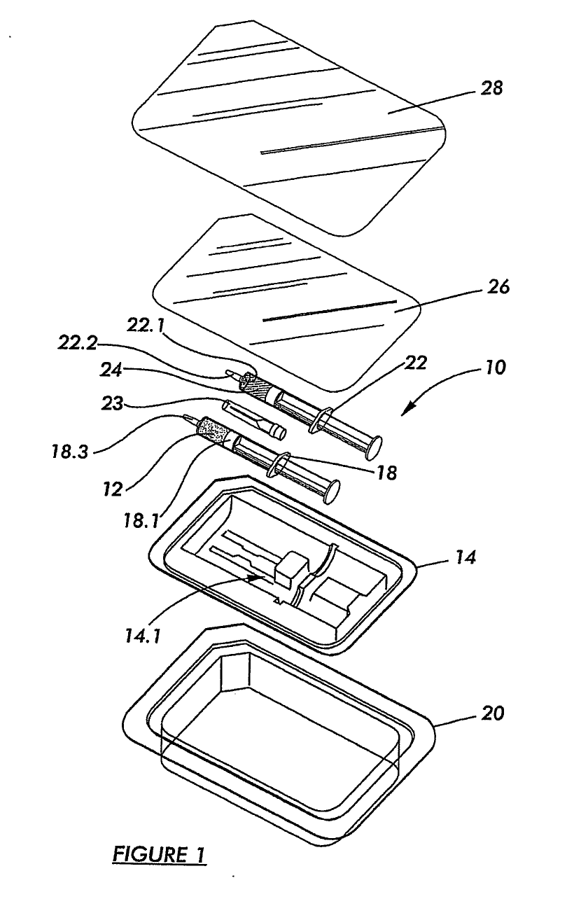

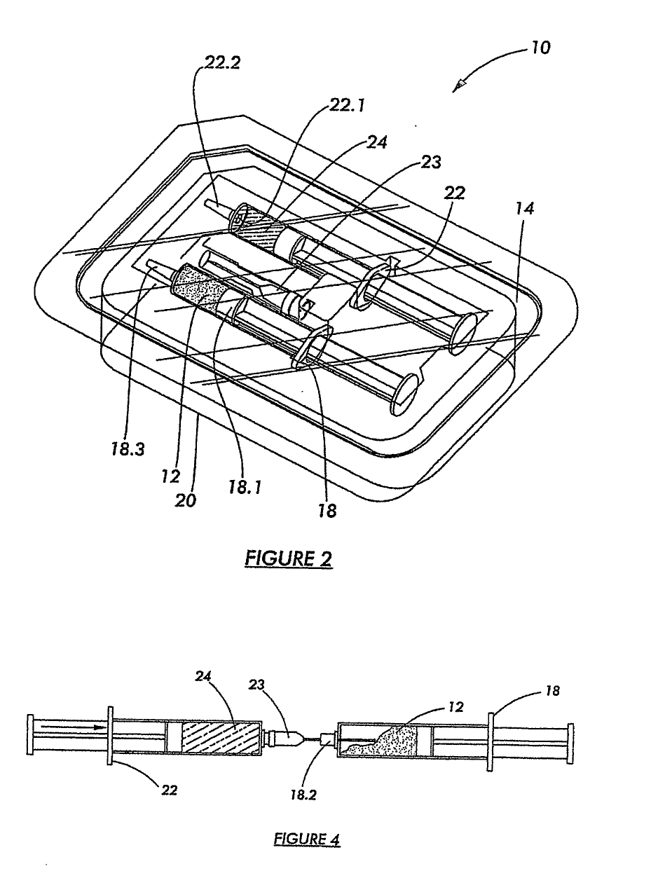

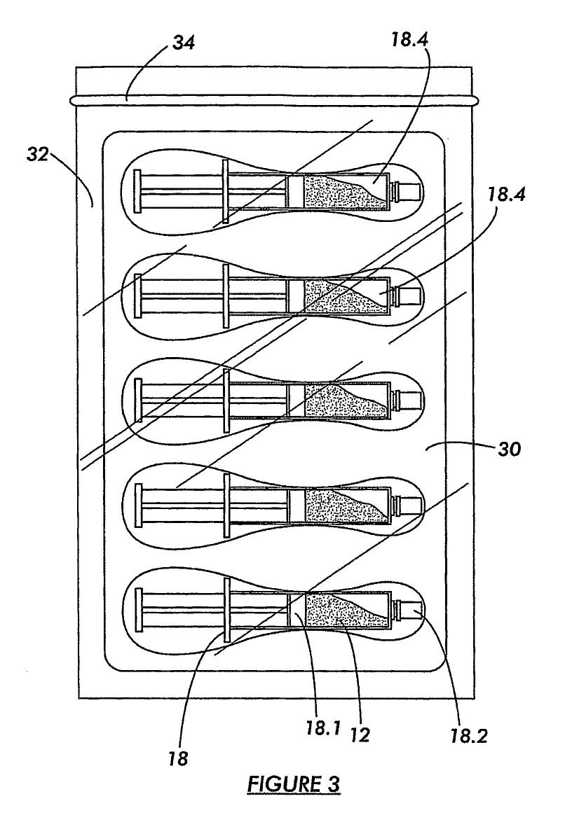

Referring to FIGS. 1 and 2, a kit according to a preferred embodiment of the invention for preparing an osteoinductive agent, is generally designated by reference numeral 10.

The kit 10 includes a modified naturally occurring biocompatible biopolymer which was subjected, in the solid, or dry state, to a source of ionising radiation in the presence of a mediating gas and annealed in the absence of oxygen at a temperature of from 40° C. to 120° C. to render the product 12 in a dry particulate form, as discussed in more detail below. The product 12 is disposed in a first primary container 18 which, in turn, is disposed in a hermetically sealed secondary container 14 containing an inert oxygen-free gas in the form of nitrogen.

The first primary container 18 is in the form of a syringe—type container of a radiation stable polymer of the type known in the art of syringe manufacturing. The container 18 is therefore provided with a plunger 18.1 for dispensing the contents thereof and an outlet opening 18.2, having a diameter larger than 0.6 mm, to allow for the dispensing of the product in a relatively viscous form. The opening 18.2 is covered with a removable cap 18.3 defining an opening (not shown) for allowing the passage of the mediating gas, as well as the said nitrogen gas into and out of the first secondary container 18. A space 18.4 in the first primary container not occupied by the product 12 is thus filled with the nitrogen gas.

The kit 10 yet further includes a similar syringe—type second primary container 22 containing a liquid in the form of pyrogen-free water 24. The second primary container 22 is also disposed inside the hermetically sealed secondary container 14. The kit 10 also includes a blunt needle 23 which fits over an outlet 22.1 of the container 22, for fitting inside the outlet opening 18.2 of the first primary container 18, in use, to Inject the pyrogen-free water 24 into the space 18.4. The outlet spout is closed with a cap 22.2.

The kit 10 further includes a hermetically sealed tertiary container 20 also filled with an inert oxygen-free gas in the form of nitrogen gas. The hermetically sealed secondary container 14 is disposed inside the tertiary container 20. The secondary and tertiary containers 14 and 20 are vacuum formed from a radiation stable, gas—impermeable material such as PET.

The secondary and tertiary containers 14 and 20 are each hermetically sealed by a peelable, radiation stable, gas—impermeable, ti-laminate cover 26 and 28 respectively. Each cover 26 and 28 comprises an aluminium layer sandwiched between an outer polyester layer and an inner polyethylene layer.

The secondary container 14 defines recesses 14.1 for receiving and releasably locating the two primary containers 18 and 22 and the needle 23.

In use, referring additionally to FIG. 3, in preparation of the kit 10, a plurality of first primary containers 18, containing the biocompatible biopolymer(s) (12) in dry or solid form are disposed in a tray 30. The tray 30 and the biopolymer containing containers 18 are disposed inside a radiation stable, gas—impermeable pouch 32 and the pouch 32 hermetically sealed with a seal 34. Just prior to sealing, the air inside the pouch 32, including the air inside the primary containers 18, is replaced with a mediating gas such as selected from the group consisting of acetylene, ethylene and propylene, to saturate the biopolymer with the mediating gas. The sealed pouch 32, whilst containing the biopolymer(s) (12), is subjected to a source of ionising radiation to obtain a minimum absorbed close of 16 kGy in the biopolymer(s) (12).

The source of ionising radiation is either a radioactive isotope such as ⁶⁰Co (γ-rays), or radiation generated by a high energy (250 keV to 10 MeV) electron accelerator, or X-rays generated by the accelerator, or any other suitable device.

The minimum absorbed radiation dose may vary from 1 kGy to 50 kGy, depending on the structure of the biopolymer and whether a branched or long-chain nature of the product is desired, but is typically 16 kGy for the biopolymers selected herein.

Following the irradiation step In the presence of the mediating gas (acetylene), and in order to remove any chemically reactive species produced by the radiation step, the irradiated biopolymer(s) (12) is/are subjected to heat treatment (annealing) in the absence of oxygen to form the product 12 in the form of a cross-linked dry particulate biopolymer. The annealing takes place at elevated temperatures ranging from 40° C. to 120° C. depending on the heat stability of the particular biopolymer which is being modified. This annealing step is ideally carried out in the presence of the acetylene or another unsaturated gaseous atmosphere or, alternatively, in the presence of an inert gas such as nitrogen or helium, or further alternatively in a vacuum oven. Annealing in the presence of acetylene could increase the formation of the new product 12, whilst annealing in vacuum or inert gas provides a suitable mechanism for the elimination of any chemically reactive free radicals formed during the process.

Following the annealing step, any residual gaseous mediating agent (acetylene) is removed from the product 12; the primary containers 18; and the pouch 32 by aerating the pouch 32, and if necessary, the application of a vacuum process to the product 12. This will depend on the retention ability of the product 12 for the gas, which depends on the porosity of the product 12.

Following the annealing step, the first primary container 18 containing the cross-linked product 12, is located in the secondary container 14. A desired amount of pyrogen—free water is disposed in the second primary container 22 and disposed in the secondary container 14 together with the needle 23. These steps take place in a nitrogen atmosphere to prevent the contact of oxygen with the product 12.

The secondary container 14 is then hermetically sealed with the cover 26 whilst capturing nitrogen gas inside the container 14. The secondary container 14 is then inserted into the tertiary container 20, preferably in a nitrogen atmosphere and the tertiary container 20 hermetically sealed with the cover 28, also capturing nitrogen gas inside the tertiary container 20, to complete the kit 10. Thereafter, the entire kit 10 is radiation sterilised by subjecting the kit 10 to a minimum absorbed irradiation dose of 25 kGy. The kit 10 can now be stored at ambient temperatures for a period of up to 5 years.

When an osteoinductive agent is to be prepared for use in reconstructive bone surgery, the secondary and tertiary containers 14 and 20 are opened by peeling open the covers 28 and 28 respectively. The caps 18.3 and 22.2 are removed from the secondary containers 18 and 22 and the needle 23 placed on the outlet 22.3 of the second primary container 22. The needle 23 is inserted into the opening 18.2 and pyrogen-free water 24 injected into the space 18.4 and mixed with the product 12. The product 12 is thus hydrated to form an osteoinductive agent in putty form. The putty is manually dispensed into a bone reconstruction site (not shown) in a human or animal body and the site closed to allow bone reconstruction to take place. It will be appreciated that these steps have to take place under aseptic conditions.

The naturally occurring biocompatible biopolymer is selected from the group consisting of collagen; hyaloronic acid; demineralised bone (DMB); and mixtures thereof. In the case of the said mixture, in preparation of the kit 10, the biocompabble biopolymers are subjected separately from each other in the presence of the said mediating gas to the said source of ionising radiation and thereafter mixed. Alternatively, in the case of the said mixture, the biocompatible biopolymers are first mixed and thereafter subjected to the said source of ionising radiation in the presence of the said mediating gas.

For example, the dry DMB and collagen are pre-mixed in the required ratio (40:60) and placed in the primary container 18. This dry mixture of the DMB and the collagen is then subsequently radiation cross-linked as herein described at the optimum minimum absorbed irradiation dose of 16 kGy, which is the same for both biopolymers.

It was found that the radiation cross-linking of collagen or hyaloronic acid in the dry form in the presence of a mediating gas results in a carrier for DMB that does not show the undesirable physiological side effects observed with prior art chemically cross-linked alternatives.

In carrying out the method for the preparation of the osteoinductive product 12, the biopolymer must be in the solid state, i.e. dry, in an atmosphere comprising a mediating agent, preferably a low molecular weight unsaturated alkenic or alkynic gas such as ethylene, propylene or acetylene. Acetylene is preferable. Before introducing the mediating gas to the space 18.4, the space must be flushed or evacuated to remove any oxygen therefrom. All the mediating gas is removed after completion of the radiation cross-linking process and therefore, the resulting product should not contain any of the mediating gas.

It was found that radiation cross-linking of DMB results in a 350% increase in the osteoinductive capacity of the DMB and the associated strength of the new bone. It was further found that the radiation cross-linking of collagen or hyaloronic acid results in a thousand fold increase in the molecular mass of the modified collagen and hyaloronic acid, thus rendering these modified biopolymers as excellent carriers for the DMB.

This method of preparing the crosslinked osteoinductive agent in accordance with the invention has Inter alia the following advantages:
The dry osteoinductive product 12 will have an elongated shelf life relatively much longer than the prior art systems, as the product 12 is stored in dry form under oxygen-free gas and the hydrated osteoinductive putty Is prepared freshly directly before use in theatre. The current need for cold storage of such osteoinductive agents is thus obviated.
Because of the blanketing of the product 12 and the other components of the kit 10 with nitrogen gas prior to the radiation sterilisation and storage, virtually no radiation-induced oxidative degradation of the dry product 12, the containers 18 and 22, and the packaging of the secondary container 14 takes place. This results in the enhanced packaging integrity and ensuing shelf—life of the product 12. The latter estimated to be at least five years at ambient temperatures.
The method of the present invention further subjects the product 12 and the pyrogen—free water 24 to a terminal radiation sterilisation process and the associated very high degree of sterility assurance and safety to the patient.

It will be appreciated that variations in detail are possible with the use and preparation of an osteoinductive agent, with an osteoinductive kit including such agent and with the use of the said kit in the dispensing of such an osteoinductive agent, according to the invention without departing from the scope of the appended claims.

1. A method for the preparation of an osteoinductive agent including the steps of modifying a naturally occurring biocompatible biopolymer by subjecting the biopolymer in the solid, or dry state, to a source of ionising radiation in the presence of a mediating gas and annealing the resulting product in the absence of oxygen at a temperature of from 40° C. to 120° C. to render the product in a dry particulate form; thereafter removing any residual mediating gas; and disposing the product in a hermetically sealed container containing oxygen-free gas.

2. A method according to claim 1 wherein the naturally occurring biocompatible biopolymer is selected from the group consisting of collagen; hyaloronic acid; demineralised bone (DMB); and mixtures thereof.

3. A method according to claim 2 wherein, in the case of the said mixture, the method includes the steps of first subjecting the biocompatible biopolymers to the said source of ionising radiation in the presence of the said mediating gas separately from each other; and thereafter mixing the irradiated biocompatible biopolymers.

4. A method according to claim 2 wherein, in the case of the said mixtures, the method includes the steps of first mixing the biocompatible biopolymers; and thereafter subjecting the mixture to the said source of ionising radiation in the presence of the said mediating gas.

5. A method according to claim 3 wherein the biocompatible biopolymers are subjected to a minimum absorbed irradiation dose of 16 kGy.

6. A method according to claim 1 wherein the hermetically sealed container is a secondary container and wherein the method includes the further step of disposing the product inside a first primary container, which is disposed inside the hermetically sealed secondary container.

7. A method according to claim 6 including the further step of providing the first primary container in the form of a syringe-type container, having a plunger for dispensing the contents thereof, and an outlet opening having a diameter larger than 0.6 mm, to allow for the dispensing of the said product in a relatively viscous form.

8. A method according to claim 7 including the further step of filling the space in the first primary container not occupied by the product with the said oxygen-free gas.

9. A method according to claim 6 which includes the further steps of providing a second primary container; disposing liquid in the second primary container; and disposing the second primary container inside the hermetically sealed secondary container.

10. A method according to claim 9 including the further step of providing the said liquid in the form of pyrogen-free water.

11. A method according to claim 9 which includes the further step of disposing the hermetically sealed secondary container inside a hermetically sealed tertiary container.

12. A method according to claim 11 including the further step of filling the tertiary container with oxygen-free gas and capturing the oxygen-free gas inside the hermetically sealed tertiary container.

13. A method according to claim 11 wherein the steps of providing the secondary and tertiary containers include the step of vacuum forming these containers from a radiation stabilised, gas-impermeable material.

14. A method according to claim 9 including the further steps of subjecting the said containers and their contents, in kit form, to a terminal radiation sterilisation process.

15. A method according to claim 14 wherein the sterilisation process includes the step of subjecting the containers and their contents to a minimum absorbed irradiation dose of 25 kGy.

16. A method according to claim 14 which includes the further step of opening the sealed containers and mixing the said sterile liquid with the said product in a dry particulate form to hydrate the product to form an osteoinductive agent in the form of a pliable viscous putty.

17. A method according to claim 16 which includes the further step of dispensing the osteoinductive agent from the first primary container to a bone reconstruction site.

18. A method according to claim 1 including the step of providing the oxygen-free gas in an inert form.

19. A method according to claim 18 including the further step of providing the said gas in the form of nitrogen.

20. A method according to claim 1 wherein the said mediating gas is selected from the group consisting of acetylene, ethylene and propylene.

21. A kit for preparing and dispensing an osteoinductive agent including a modified naturally occurring biocompatible biopolymer which was subjected, in the solid, or dry state, to a source of ionising radiation in the presence of a mediating gas and annealed in the absence of oxygen at a temperature of from 40° C. to 120° C. to render the product in a dry particulate form, the product being disposed in a hermetically sealed container containing oxygen-free gas.

22. A kit according to claim 21 wherein the naturally occurring biocompatible biopolymer is selected from the group consisting of collagen; hyaloronic acid; demineralised bone (DMB); and mixtures thereof.

23. A kit according to claim 22 wherein, in the case of the said mixture, the biocompatible biopolymers are subjected in the presence of the said mediating gas to the said source of ionising radiation separately from each other and thereafter mixed.

24. A kit according to claim 22 wherein, in the case of the said mixture, the biocompatible biopolymers are first mixed and thereafter subjected to the said source of ionising radiation in the presence of the said mediating gas.

25. A kit according to claim 23 wherein the biocompatible biopolymers are subjected to a minimum absorbed irradiation dose of 16 kGy.

26. A kit according to claim 21 wherein the sealed container is a secondary container and wherein the product is disposed inside a first primary container, which is disposed inside the sealed secondary container.

27. A kit according to claim 26 wherein the first primary container is in the form of a syringe-type container, having a plunger for dispensing the contents thereof and an outlet opening having a diameter larger than 0.6 mm, to allow for the dispensing of the product in a relatively viscous form.

28. A kit according to claim 26 wherein the space in the primary container not occupied by the product is filled with the said oxygen-free gas.

29. A kit according to claim 26 which includes a second primary container containing a liquid and being disposed inside the hermetically sealed secondary container.

30. A kit according to claim 29 wherein the liquid is in the form of pyrogen-free water.

31. A kit according to claim 29 wherein the hermetically sealed secondary container is disposed inside a hermetically sealed tertiary container.

32. A kit according to claim 31 wherein the tertiary container is filled with oxygen-free gas.

33. A kit according to claim 32 wherein the secondary and tertiary containers are vacuum formed from a radiation stable, gas-impermeable material.

34. A kit according to claim 29 wherein the said containers are subjected, in kit form, to a terminal radiation sterilisation process.

35. A kit according to claim 21 wherein the oxygen-free gas is inert.

36. A kit according to claim 35 wherein the gas is nitrogen.

37. A kit according to claim 21 wherein the said mediating gas is selected from the group consisting of acetylene, ethylene and propylene.

38. A method of reconstructive bone surgery in humans or animals including the steps of providing the kit in according to claim 34; opening the secondary and tertiary containers; hydrating the dry particulate product by injecting the sterile liquid into the first primary container and mixing the liquid and the product to form a putty; dispensing the putty into a bone reconstruction site; and closing the site to allow bone reconstruction to take place.

39. An osteoinductive agent prepared in accordance with claim 1.

40. (canceled)

41. (canceled)
